(19) United States
(12) Patent Application Publication
(10) Pub. No.: US 2022/0249724 A1
(43) Pub. Date: Aug. 11, 2022

(54) Universal Wing Adapter Assembly for Holding Surgical Instruments

(71) Applicant: Benjamin Haak, Park Ridge, IL (US)

(72) Inventor: Benjamin Haak, Park Ridge, IL (US)

(21) Appl. No.: 17/397,773

(22) Filed: Aug. 9, 2021

Related U.S. Application Data
(63) Continuation-in-part of application No. 17/170,527, filed on Feb. 8, 2021, now abandoned.

(51) Int. Cl. A61L 2/26; A61B 90/70

(52) U.S. Cl. CPC A61L 2/26; A61L 2202/122; A61L 2202/24; A61B 90/70

(57) ABSTRACT

A universal wing adapter assembly holds surgical instruments in a sterilization container during a sterilization process in an autoclave or other sterilization machine. The universal wing adapter assembly is configured to fit on multiple predefined patterns of holes found on the floor of a sterilization container. The universal wing adapter assembly may be rotated, and/or slid back and forth, in order to fit various sized surgical instruments in the sterilization container, thus enabling the sterilization container to be loaded to capacity.

This application claims priority from and incorporates by reference in its entirety U.S. patent application Ser. No. 17/170,527 filed on Feb. 8, 2021.

### BACKGROUND

### Technical Field

Various embodiments of the present invention relate to surgical instruments, and more specifically, to equipment for sterilizing surgical instruments of various sizes and shapes.

### Description of Related Art

It is crucial that surgical tools are thoroughly cleaned and sterilized after each use to prevent the possibility of introducing a contaminant from one patient to the next. This is done by placing the surgical tools in a sterilization atmosphere for one or more cycles of predetermined lengths. One typical sterilization atmosphere is provided in an autoclave that bathes the surgical tools in steam. Other sterilization chambers provide a gaseous chemical sterilization atmosphere such as ethylene oxide. Dry heat sterilization may also be used in some situations.

FIG. 1A depicts a prior art surgical tool holder bar 101 that is used to hold a surgical tool while it is being bathed in the sterilization atmosphere. FIG. 1B depicts two prior art surgical tool holder bars 101 mounted in a rectangular sterilization basket 101. Surgical tools are typically fastened to reusable sterilization containers 101 which are placed into the sterilization chamber. Surgical tool holder bars 101 hold the surgical tools up away from the floor of the sterilization container 101 to fully expose them to the sterilization atmosphere and keep them from clanging around during the sterilization procedure itself, while putting the surgical tools into and taking them out of the sterilization chamber, and while transporting the sterilization container 101 from one place to another.

Various types of sterilization containers are in use today, including wire mesh baskets and trays with a preconfigured array of holes 105 for ventilation and attaching the surgical tool holder bars 101. The baskets or trays are typically made out of stainless steel or other durable material and tend to cost a fair amount—often in excess of $100 each. Conventional surgical tool holder bars 101 are fastened to the sterilization container 101 with screws 107 that fit through the preconfigured array of holes 105.

### SUMMARY OF THE INVENTION

The present inventor recognized a major drawback of conventional instrument bar assemblies used in standard sterilization containers inasmuch as they cannot readily be adapted to accommodate new or additional instruments in the containers for which they were designed. This is due in part to conventional instrument bars being designed to be used in connection with the preconfigured arrangements of holes in a particular brand of sterilization container. Given the cost of sterilization containers, and the storage space required to store specialized sterilization containers for each type and size of surgical instrument, the present inventor recognized the efficiencies to be realized in being able to reconfigure the surgical instrument holder bars to accommodate the tray hole positions for the numerous sizes and shapes of various surgical instruments. Various embodiments of the present invention overcome these drawbacks and provide other benefits and advantages, as disclosed below.

### BRIEF DESCRIPTION OF THE DRAWINGS

The accompanying drawings, which are incorporated in and constitute part of the specification, illustrate various embodiments of the invention. Together with the general description, the following drawings serve to explain the principles of the invention.

FIG. 1A depicts a prior art surgical tool holder bar.

FIG. 1B depicts a prior art surgical tool holder bar mounted in a rectangular sterilization basket.

FIG. 2A depicts two multi-tool instrument bars mounted on universal wing adapter assemblies within an adjustable sterilization tray, according to various embodiments.

FIG. 2B depicts six multi-tool instrument bars mounted on universal wing adapter assemblies for holding surgical tools mounted within a wire mesh sterilization basket, according to various embodiments.

FIG. 3A is an oblique view of a base rail unit of a universal wing adapter assembly for holding surgical tools, according to various embodiments.

FIG. 3B depicts two views of a universal wing adapter assembly that includes the base rail unit of FIG. 3A with an adjustable bar track in different positions relative to the base rail unit, according to various embodiments.

FIG. 3C is a side view of an instrument holder bar suitable for mounting on a universal wing adapter assembly for holding surgical tools, according to various embodiments.

FIG. 3D is an oblique view of an adjustable bar track configured to receive an instrument holder bar, according to various embodiments.

FIG. 4A depicts a top view of a universal wing adapter assembly base rail unit, according to various embodiments.

FIG. 4B depicts a front view of a universal wing adapter assembly base rail unit, according to various embodiments.

FIG. 4C depicts a side view of a universal wing adapter assembly base rail unit, according to various embodiments.

FIG. 4D depicts an oblique side view of an adjustable bar track mounted on a universal wing adapter assembly base rail unit, according to various embodiments.

FIG. 4E depicts an oblique view of a universal wing adapter assembly base rail unit with a mounting nut and mounting bolt, according to various embodiments.

FIGS. 5A-B depict an instrument holder bar fitting onto an adjustable bar track, and a surgical instrument being held by an instrument holder bar, according to various embodiments.

FIG. 6A depicts a top view of a universal wing adapter assembly base rail unit with a slotted hole, according to various embodiments.

FIG. 6B depicts an oblique view of the base rail unit with a slotted hole, according to various embodiments.

FIG. 7 depicts a top view of a universal wing adapter assembly base rail unit 711, according to various embodiments.

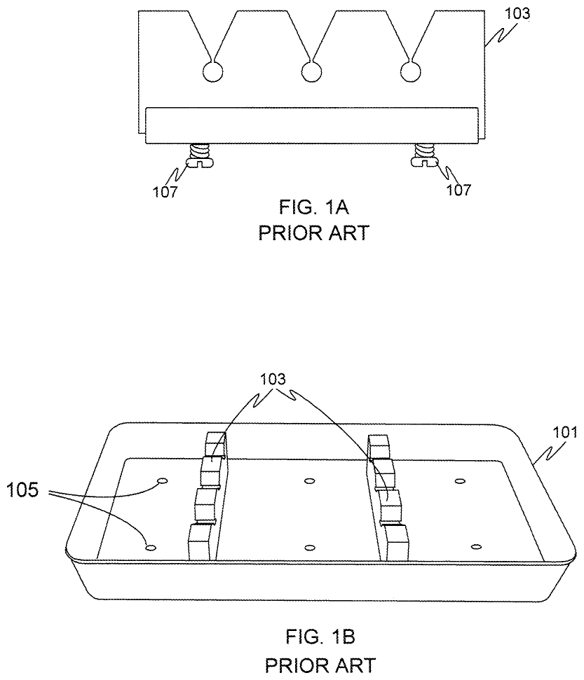

### DETAILED DESCRIPTION

The present inventor recognized several advantages and benefits from being able to vary the positions of surgical instrument holder bars within a sterilization basket. Since sterilization baskets have predefined hole patterns for mounting conventional surgical tool holder bars, the arrangement of tools within them is quite limited. The present inventor recognized that conventional instrument bars with fixed attachment mechanisms generally won't fit into sterilization containers that they weren't designed for due to the prearranged hole patterns of the sterilization containers. The conventional surgical tool holder bars will only fit on the predefined hole patterns in the manner intended by the manufacturer. Surgical tools typically cannot be laid crossways or diagonally with the conventional surgical tool holder bars. The present inventor recognized that by being able to reposition the surgical instrument holders according to the various embodiments, the hospital staff or other person preparing the sterilization container can load it with a set of surgical tools of different sizes and shapes from various manufacturers. This is useful inasmuch as it allows more surgical tools to be sterilized in each batch, and also because surgical tool sets chosen for a given procedure or operation can be sterilized all at once, rather than requiring multiple batches in the sterilization chamber. Further, by utilizing the various embodiments disclosed herein the hospital won't need to keep as many sterilization containers on hand since the various embodiments allow surgical tools to be placed in nearly any sterilization basket.

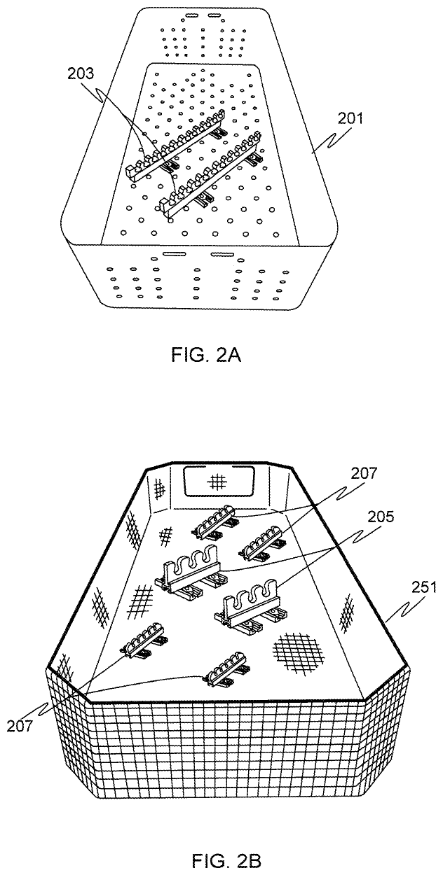

At first glance it might seem like drilling more holes in the sterilization container would afford a simple solution to the drawbacks mentioned above. This is not the case, however, since the holes in the sterilization container must typically be treated to avoid corrosion. The holes may be painted, chromed, anodized or otherwise treated to resist corrosion over time. Simply drilling a new pattern of holes in a metal sterilization container would likely result in corrosion or rust after a number of cycles through the sterilization process, which would lead to the introduction of impurities into the sterilization atmosphere. Rust and corrosion aren't a problem with plastic sterilization container. But the plastic sterilization containers are not as sturdy to begin with. So only a few holes could be drilled in a given plastic sterilization container, and each drilled hole would further weaken its structural integrity. One the other hand, a wire mesh basket as shown in FIG. 2B has a great number more holes than a basket with a predefined pattern of holes. But even the larger number of holes in the wire mesh basket may not be in the right place to position the surgical instruments in the manner desired. The wire of a wire mesh basket can be bent slightly to move an instrument a few degrees one way or another, but bending the wires may result in the chrome or other coating flaking off the wires which in turn introduces impurities into the sterilization atmosphere.

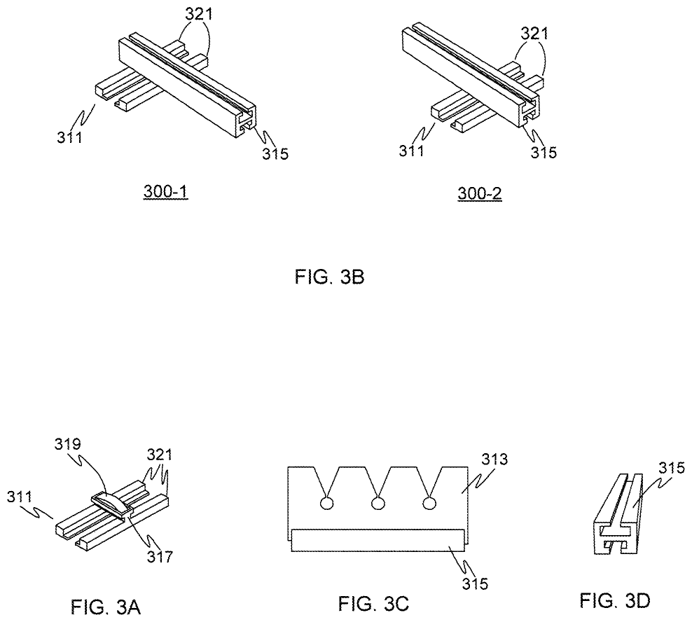

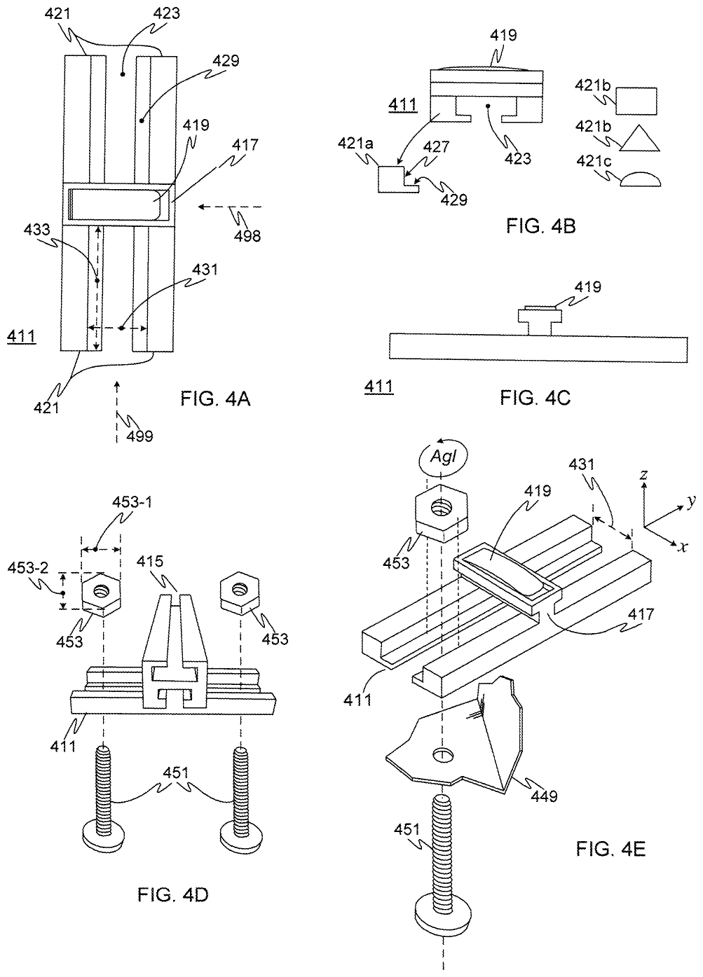

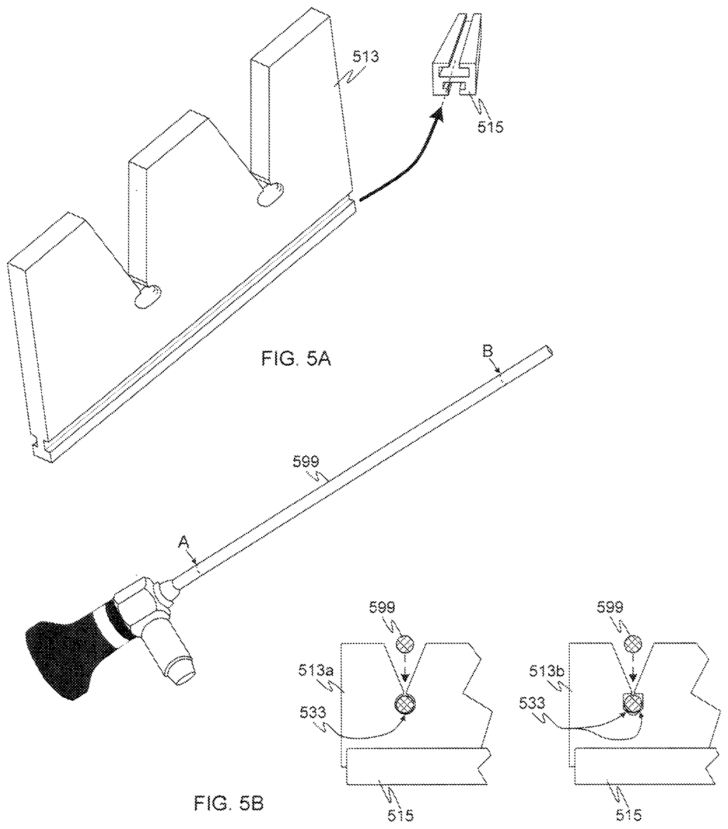

FIGS. 2A and 2B depict sterilization containers that are equipped with some of the different types of surgical tool holder universal wing adapter assemblies 203/205/207, according to various embodiments. The sterilization containers may sometimes be called sterilization baskets or sterilization trays. The surgical tool holder universal wing adapter assembly may sometimes be called a universal adapter assembly. FIG. 2A depicts two universal wing adapter assemblies 203 adjustably mounted within an adjustable sterilization tray 201, according to various embodiments. FIG. 2B depicts four universal wing adapter assemblies 207 and two universal wing adapter assemblies 205 adjustably mounted within a wire mesh sterilization basket 251. The figure illustrates the flexible placement capabilities of the universal wing adapter assemblies in a sterilization container. In practice several more universal wing adapter assemblies could be fit into the sterilization container in order to more fully load it up for a sterilization process in an autoclave or other sterilization machine. Details of the universal wing adapter assembly are discussed in conjunction with FIGS. 3A-D and FIGS. 4A-5A.

FIG. 3A depicts a base rail unit 311 of a universal wing adapter assembly for holding surgical tools, according to various embodiments. FIG. 3B depicts two views of a universal wing adapter assembly that includes the base rail unit 311 of FIG. 3A with an adjustable bar track 315 in different positions relative to the base rail unit 311, according to various embodiments. FIG. 3C depicts an instrument holder bar 313 and adjustable bar track 315 suitable for mounting on a base rail unit 311 of a universal wing adapter assembly for holding surgical tools, according to various embodiments. FIG. 3D depicts an adjustable bar track 315 configured to receive an instrument holder bar 313, according to various embodiments. Together the base rail unit 311 and the adjustable bar track 315 form a universal wing adapter assembly for securely holding an instrument holder bar 313 in a sterilization container. As such, the lower side of base rail unit 311 fits on the floor of the sterilization container. Since the floor of the sterilization container is typically flat—or has a mesh area that is flat—at least a portion of the lower side of base rail unit 311 may be flat as well so as to fit on the floor of the sterilization container. It should be noted that the contour of the lower side of base rail unit 311 is not required to precisely match the contour of the floor of the sterilization container in order for the device to “fit” on the sterilization container floor. The instrument holder bar 313 is configured to hold surgical tools—e.g., 303/305—while they are being sterilized in a sterilization chamber and while transporting the sterilization container from one place to another.

The base rail unit 311 is configured to sit on the floor of a sterilization container such as a sterilization basket 301 of FIG. 2A. The base rail unit 311 includes a track rail 317 positioned towards its top—that is, mounted on its upper surface of the base rail unit 311. It may be that some implementations have portions of the base rail unit 311 that extend above where the track rail 317 is positioned (not shown). But typically the track rail 317 is positioned towards the top of the base rail unit inasmuch as the track rail 317 holds the adjustable bar track 315 high enough not to collide with any other portion of the universal wing adapter assembly. The adjustable bar track 315, which holds instrument holder bar 313, is slidably mounted on the track rail 317. This allows the position of adjustable bar track 315 to be adjusted by sliding it either way on the track rail 317 of the base rail unit 311. For example, in FIG. 3B the universal wing adapter assembly shown in view 300-1 (shown without an instrument holder bar 313) has its adjustable bar track 315 pushed towards the right with respect to base rail unit 311. Universal wing adapter assembly in view 300-2 has its adjustable bar track 315 pushed towards the left with respect to base rail unit 311. In this embodiment the base rail unit 311 includes two base rail arms 321, each of which extends outward in both directions from the track rail 317 as shown in FIG. 3A. In other embodiments the base rail unit 311 can have one base rail arm 321 rather than two.

The track rail 317 is fastened, mounted, or otherwise affixed, to the base rail unit 311. In some implementations the track rail 317 is fastened to the base rail unit 311 by being formed (e.g., machined or molded) from the same piece of material as the base rail unit 311. In various embodiments the track rail 317 has a tensioner component 319 mounted on it. In some embodiments the tensioner component 319 includes a spring-loaded button-like part that pushes upward against the inner portion of the slotted hole of adjustable bar track 315, creating friction between the two parts causing the adjustable bar track 315 to tend to stay in place relative to the track rail 317 yet retain adjustability. The spring may be a coil spring, a leaf spring, or other type of spring known to those of ordinary skill in the art. In other embodiments, a flexible piece of plastic, rubber, metal or other like type of elastic material may be used as the spring to produce the force that pushes on the adjustable bar track 315, tending to keep it in place. In this way, the adjustable bar track 315 can still be adjusted by sliding it back and forth on the track rail 317. The friction between the two components caused by the tensioner component 319 tends to keep the adjustable bar track 315 in place once it has been positioned.

FIG. 3A depicts the tensioner component 319 positioned on top of track rail 317. However, in other implementations the tensioner component 319 may be positioned on one or both sides of track rail 317. In yet other implementations the tensioner component 319 may be within the slotted hole of adjustable bar track 315. In various embodiments the parts of the tensioner component 319 are made from materials that are impervious to the harsh conditions of sterilization chambers.

The universal wing adapter assemblies 203, 205 and 207 of FIGS. 2A-B differ from each other mostly in the design of the instrument holder bar that is attached to each of the various universal wing adapter assemblies. FIG. 3C shows instrument holder bar 313 which is one of the many types of instrument holder bars that are suitable for use with the various embodiments. The instrument holder bar 313 is typically made from a flexible material that is heat resistant and/or resistant to the chemicals in the sanitizing atmosphere. For example, various embodiments of the instrument holder bar 313 may be made from silicone since it is heat resistant, flexible, and resistant to the chemicals often used to sanitize surgical instruments. The shape and hole sizes of the various implementations of instrument holder bars 313 may be tailored to hold different types of surgical instruments, e.g., scalpels, scissors, blades, forceps, retractors, endoscopes, cannulas or any other instrument or tool used for medical procedures or requiring sterilization. The instrument holder bar 313 is affixed to adjustable bar track 315.

FIG. 4A depicts a top view of a universal wing adapter assembly base rail unit 411, according to various embodiments. FIG. 4B depicts a front view (looking in direction 499 of FIG. 4A) of the universal wing adapter assembly base rail unit 411, according to various embodiments. FIG. 4C depicts a side view (looking in direction 498 of FIG. 4A) of the universal wing adapter assembly base rail unit 411, according to various embodiments. FIG. 4D depicts an oblique side view of an adjustable bar track 415 mounted on the base rail unit 411, according to various embodiments. The base rail unit 411 includes a track rail 417 with a tensioner component 419. The adjustable bar track 415—which holds instrument holder bar (e.g., 513 of FIG. 5A and 313 of FIG. 3C)—is configured to slide back and forth in direction 498 on the track rail 417 of the base rail unit 411. The tensioner component 419 presses against the adjustable bar track 415, keeping it in place once it has been adjusted by the user. The amount of force exerted by the tensioner component 419 on the adjustable bar track 415 can vary depending upon the requirements of the implementation, but is generally at least 0.05 lbs. of force and may be up to 40 lbs of force.

FIG. 4E depicts an oblique view of the universal wing adapter assembly base rail unit 411 with mounting nut 453 and mounting bolt 451, according to various embodiments. The mounting bolt 451 is a type of threaded attachment mechanism. Other types of threaded attachment mechanisms may be used to fasten the universal wing adapter assembly to the floor of a sterilization container. The figure shows mounting nut 453 embodied as a hex nut. In practice, however, the various embodiments may be implemented using various different types of threaded nuts including square nuts, rectangular nuts, wing nuts, or even nuts with a round outer cross-sectional shape (that are typically knurled to allow a user to grasp the nut) or other type of threaded nut known to those of ordinary skill in the art. In various embodiments, the mounting nut 453 fits down into the trough area which is bounded by the two L-shaped base rail arms 421a of base rail unit 411 as shown in FIGS. 4B and 4E. In this respect, the trough area between the two base rail arms 421 accepts the mounting nut 453, preventing it from turning as the mounting bolt 451 is screwed into it from below.

The mounting nut 453 is characterized by a distance between flats parameter 453-1 and a distance between points parameter 453-2. Other types of female threaded attachment mechanisms may also be characterized by a distance between flats parameter and a distance between points parameter as well. The “flats” (i.e., flat sides) of the female threaded attachment mechanisms are where the surfaces of a wrench lit to loosen/tighten the nut (or other female threaded attachment mechanism). The distance between points for a square (or rectangular) nut is the distance between opposite corners. Typically, the flats of the female threaded attachment mechanism fit against the trough side walls 427—one on each side, preventing the female threaded attachment mechanism (e.g., mounting nut 453) as the male threaded attachment mechanism (e.g., mounting bolt 451) is screwed into it. The points are formed by two adjacent flats, or at the edges of a flat that is positioned against the trough side walls 427. The trough walls are separated by a trough width 431 that is greater than the distance between flats 453-1—that is, slightly greater than the width between the two parallel flat sides of the mounting nut 453—but less than the distance between points parameter of mounting nut 453. Typically, the trough width 431 is at least 0.01 inch longer than the distance between flats 453-1, and the trough width 431 is closer in length to the distance between flats 453-1 than to the distance between points 453-2. The trough length 433 is typically somewhat longer than the trough width 431. FIG. 4A depicts a trough length 433 that is slightly more than twice as long as the trough width 431. In some embodiments the trough length 433 is at least as long as the trough width 431. In other embodiments the trough length 433 is at least twice as long as the trough width 431.

The trough floor 429 and adjacent trough side wall 427 of each L-shaped base rail arm 421a creates a rabbet profile slot 423 that fits along a side of the mounting nut 453 and portion of the bottom of the mounting nut 453. The trough floor 429 serves as a base portion of the base rail arm 421 for the mounting nut 453 (or other threaded attachment mechanism) to be tightened against. As the mounting bolt 451 is tightened it causes the mounting nut 451 to press against the trough floor 429 of base rail arm 421, securely (but removably) fastening it to the floor of the sterilization chamber. In this sense the trough floor 429 serves as a base portion of base rail arm 421 for the mounting nut 453 to be tightened against. The length of the base portion of base rail arm 421 is the same as the trough length 433.

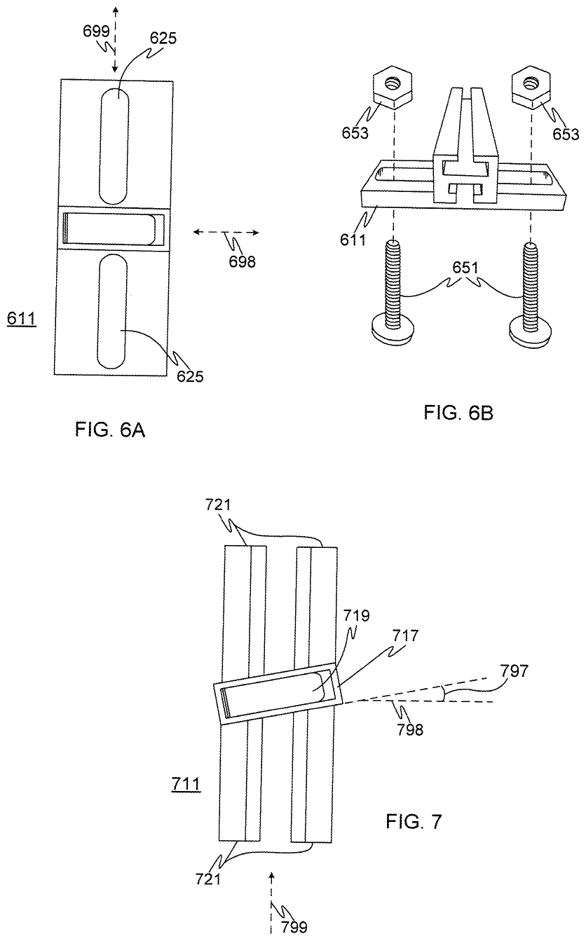

In other embodiments the base rail arms may be embodied in a rectangular cross-section 421b, a triangular cross-section 421c, a semi-circular cross-section 421c, or any other shaped cross-section known to those of ordinary skill in the art. The only limiting factor in the cross-sectional shape of the base rail arms is that the underside of the nut 453, upon making contact with the arms and upon being tightened down, creates a sufficient amount of friction so as to hold the base rail unit 411 firmly against the floor of the sterilization container. In some embodiments the arms of the base rail unit 411 may not be open at the ends as seen in the top view of FIG. 4A. Instead, the base rail unit 411 may be provided with a slotted hole, for example, as illustrated in FIGS. 6A-B by slotted hole 625. One advantage of using a trough shaped hole (e.g., with L-shaped cross-sections 421a) for mounting bolt 451 is that the trough walls 427 of the trough shaped hole keep the mounting nut 453 from turning as it is being tightened against the trough floor 429, or loosened. Similarly, in some implementations the slotted hole 625 shown in FIG. 6A may be stepped in order to keep the mounting nuts 653 from turning as they are being tightened.

The base rail unit 411 can be removably fastened within a sterilization container by placing the mounting bolt 451 through one of the holes of the sterilization container 449. The base rail unit 411 can be fastened using either one mounting bolt 451 as shown in FIG. 4E, or by using two mounting bolts 451 and mounting nuts as shown in FIG. 4D. If two mounting bolts 451 are used the base rail unit 411 can be adjusted back and forth along line 499 (shown in FIG. 4A) by adjusting the position of mounting bolts 451 within the rabbet profile slot 423 formed by the two base rail arms 421. The mounting bolt(s) 451 can be inserted into different holes in the sterilization basket 449 to orient the base rail unit 411 in various angular directions Agl as shown in FIG. 4E. If only one mounting bolt 451 is used to fasten the base rail unit 411 within the sterilization container, the base rail unit 411 can be pivoted around the mounting bolt 451 in any angular direction Agl. In addition, the base rail unit 411 can be slid back and forth along the base rail arm(s) 421 relative to the sterilization container. The adjustable bar track 415 which holds the instrument holder bar can be adjusted in any direction in the x-y plane shown in FIG. 4E, and oriented at any angular direction Agl in order to provide flexibility in loading up the sterilization container with surgical instruments to be sterilized. Thus, the various embodiments provide three degrees of planar positional freedom in positioning an instrument holder bar for holding surgical tools. The three degrees of planar positional freedom are: adjustment to different positions within a sterilization container in the x direction, the y direction and adjustment in orienting the device in any angular direction Agl in the x-y plane.

To removably fasten a universal wing adapter assembly to a sterilization container the technician (or other user) selects a hole in the sterilization container though which the first mounting bolt 451 (or other mechanical tightening mechanism such as a machine screw) is placed. The mounting bolt 451 extends through the gap between the base rail arms 421 of base rail unit 411. In the embodiments depicted in FIGS. 4A-E the gap between the rail arms is a rabbet profile slot 423. Once the base rail unit 411 is removably attached to a sterilization container with a single mounting bolt 451, the base rail unit 411 (and the rest of the universal wing adapter) can be adjusted in two manners of adjustment relative to the sterilization container. First, the base rail unit 411 can be rotated about the mounting bolt 451 extending through the hole in the sterilization container. Second, the base rail unit 411 can be slid back and forth relative to the hole in the sterilization container, putting the mounting nut 453 in different positions within the rabbet profile slot 423. Adjusting the base rail unit 411 in these two manners allows the user to select from a number of different holes in the sterilization container through which to place the second mounting bolt 451 (if desired). The universal wing adapter assembly can additionally be adjusted in a third manner once the base rail unit 411 is tightened to the sterilization container floor: By moving the adjustable bar track 415 back and forth on the track rail 417. In this way, the universal wing adapter assembly can be adjusted in three different manners.

FIG. 5A illustrates how an instrument holder bar 513 fits onto an adjustable bar track 515. FIG. 5B depicts a rigid endoscope as one example of a surgical instrument 599 which is sterilized in a sterilization chamber. FIG. 5B shows the surgical instrument 599 being held by the instrument holder bar 513, according to various embodiments. Instrument holder bars 513 come in many different sizes and shapes in order to hold the many different sizes and shapes of surgical instruments to be sterilized. The instrument holder bar 513 may be made from a flexible material in order to snap the surgical instrument 599 into place so it is securely held.

Typically, in order to ensure that the surgical instrument 599 doesn't rattle around during the sterilization process, it is held at two points. For example, the rigid endoscope surgical instrument 599 could be held at point A and point B by two different universal wing adapter assemblies. Alternatively, an instrument holder bar 513 with a larger hole could be used to hold the handle rather than at point A. In some implementations (e.g., for lighter instruments) a surgical instrument a surgical instrument may be held at only one point.

FIG. 6A depicts a top view of a universal wing adapter assembly base rail unit 611, according to various embodiments. FIG. 6B depicts an oblique view of the base rail unit 611. This embodiment of base rail unit 611 features slotted holes 625 rather than a rabbet profile slot 423 as shown in FIGS. 4A-E. The slotted hole 625 may have vertical walls from tie top to the bottom of the base rail unit 611, as shown in FIGS. 6A-B. In other embodiments, the slotted hole 625 may have a stepped wall with a profile similar to the rabbet profile slot 423 of FIGS. 4A-E in order to keep the mounting nuts 653 from turning as they are tightened.

FIG. 7 depicts a top view of a universal wing adapter assembly base rail unit 711, according to various embodiments. FIGS. 4A-E features a track rail 417 oriented perpendicular to the base rail arms 421 of the base rail unit 411. However, some embodiments feature the rail arms oriented at an angle other than perpendicular to the track rail. For example, FIG. 7 depicts track rail 717 skewed by track rail angle 797. As such the track rail 717 is oriented at a track rail angle 797 away from perpendicular to the base rail arms 721. In various embodiments the track rail angle 797 may be as little as 0 degrees up to as much as 45 degrees in either direction (positive or negative track rail angles 797), or any given angle in between 0 and 45 degrees. In one typical embodiment the track rail angle 797 is from 10 to 30 degrees (or from −10 to −30 degrees).

The different universal wing adapter assembly components of the various embodiments may be made out of any of several types of materials. For example, the universal wing adapter assembly components are typically constructed out of a high-performance thermoplastic such as Radel™, but may instead be made from a polysulfone or polyetherimide such as Ultem™, or a metal such as aluminum, brass or stainless steel. The universal wing adapter assembly components may be made out of any number of materials so long as they resist corrosion and are not otherwise subject to damage due to repeated cycles in the sterilization atmosphere, and have sufficient structural strength, as such materials are known to those of ordinary skill in the art.

The terminology used herein describes the embodiments outlined in this specification, and is not intended to limit the invention. Terms relating to direction are to be interpreted in terms of directions relative to the sterilization container to which the various embodiments of the universal wing adapter assemblies are to be attached. The floor of the sterilization container is assumed to be horizontally flat relative to the earth, so the terms “up” or “upward” refer to a direction tending away from the center of the earth. The terms “directly up” or “directly upward” refer to the direction straight upward away from the center of the earth. Despite the present discussion which assumes the sterilization container will lie horizontally flat relative to the earth, the various embodiments may be affixed to a sterilization container that is oriented at any other angle; e.g., vertical, so as to be inserted into the sterilization chamber in a rack-type configuration. The phrase “360 degree planar adjustment,” as used herein, means that the position of the device can be adjusted to be oriented in any direction (i.e., 360 degrees). The phrase “three degrees of planar positional freedom”, as this phrase is used herein, means that the device can be adjusted to different positions in the x direction and in the y direction (e.g., within a sterilization chamber), and can be oriented in any angular direction Agl. Alternatively stated, “three degrees of planar positioning freedom” constitutes two linear degrees of positional freedom in a plane (e.g., ability to change position in the x-y plane) plus angular positional freedom of position (e.g., ability to rotate so as to orient in any direction).

A given part that is “slidably mounted” on another component, as this term is used herein, is able to slide back and forth on the component. For example, the rip fence of a table saw is slidably mounted on the table of the table saw in order to adjust the cut width. The adjustable bar track 315 is configured to slide back and forth on the track rail 317 as shown in FIGS. 3A-B. As such, the adjustable bar track 315 is slidably mounted on the track rail 317 of base rail unit 311. The phrase “adjustably mounted,” as used herein, means that the device is capable of being adjusted to various positions. An “instrument mounting point,” as this term is used herein, is the lowest point on a surgical instrument holder bar that supports the surgical instrument. A “surgical instrument,” as this term is used herein, includes scalpels, scissors, blades, forceps, retractors, tweezers, endoscopes, cannulas, surgical cameras, kidney pans, organ pans, surgical trays or containers, or any other instrument, tool or device used for medical procedures or otherwise requiring sterilization in a sterilization atmosphere as are known by those of ordinary skill in the art. A “sterilization atmosphere” is the atmosphere within a sterilization chamber intended to kills germs. Typical sterilization atmospheres may include any of: steam, dry heat, gaseous chemical atmospheres such as ethylene oxide, high intensity ultraviolet light (or other light bandwidths), laser chambers, biological agents, or the like as are known by those of ordinary skill in the art. The term “sterilization chamber” includes the chamber of an autoclave or other sterilization machine, and is typically configured to receive surgical instruments and expose them to a sterilization atmosphere during a sterilization process.

The phrases “removably fastened”, “removably attached”, “removably affixed” and “removably mounted”, as used herein, mean a part (or mechanism, component device, unit etc.) that can be attached to another part, and then later removed without destroying or damaging either of the parts or the mechanism for removably attaching the two pieces. For example, a king bolt can be used to removably attach a wagon tongue to a team of horses. In regards to the various embodiments, a threaded nut and machine screw (or bolt) can be used to removably fasten a universal wing adapter assembly to a sterilization container. However, one piece of metal welded onto another piece of metal is not removably attached. Also, one part that is riveted onto another part is not considered to be removably attached since the rivets must be destroyed to separate the two parts—even though the two attached parts themselves may not be damaged. Two parts that are “permanently attached” (or “permanently fastened”, “permanently affixed” or “permanently mounted”), as used herein, are attached in a manner that is not conducive to separating the parts without damaging one part or the other, or damaging the means of attaching them together. Two parts may be “permanently attached” (or “permanently affixed”), for example, by being welded, glued or riveted together. Further two parts that are formed from the same piece of material are considered to be permanently attached together. For example, implementations of adjustable bar track 415 of FIG. 4D that are formed from the same piece of material as universal wing adapter assembly base rail unit 411 are considered to be permanently attached (or permanently affixed).

The terms “arranging” and “forming”, as these terms are used herein, means either attaching to, or forming on. For example, a vise with serrated jaw surfaces may have the serrated jaw surfaces formed on the vise jaws or the vise may have serrated jaw surface plates attached to the vise jaws. Similarly, a track rail arranged on a base rail unit can either be attached (e.g., glued, welded, riveted) or can be formed from the same piece of material. The term “accept”, as this term is used herein in regards to two components, means that one component can fit at least partially in a pocket, groove, hole or other type of depression within the other component. For example, a glove accepts a hand, a holster accepts a pistol, and a pair of base rail arms accepts a mounting nut. The tensioner component 319 of FIGS. 4A and 4E pushes against the adjustable bar track 315, causing it to tend to stay in place yet retain adjustability. The phrase “tend to stay in place yet retain adjustability” means that the part (e.g., adjustable bar track 315) stays in place while the sterilization tray is tilted up to 30 degrees in any direction, but can be adjusted by a human user applying a small amount of force to it (e.g., 0.25 pound of force or more).

A “mechanical tightening mechanism” is an apparatus that tightens one component to another. Examples of mechanical tightening mechanism include: C-clamps, spring steel binder clips, zip ties, nuts and bolts, ratchet straps, reusable twist ties, tie down straps, and other like types of mechanical tightening mechanism known to those of ordinary skill in the art. A “threaded attachment mechanism” as used herein is a type of mechanical tightening mechanism with threads to tighten/loosen it. Examples of threaded attachment mechanisms include a bolt, a machine screw, a screw, a threaded rod, or other like type of elongated part with threads configured to be screwed into a threaded nut, a threaded hole or other hole or other components with threads for tightening/loosening as are known by those of ordinary skill in the art. A bolt or machine screw is a male threaded attachment mechanism. A nut or bolt hole is a female threaded attachment mechanism. A nut (female) is said to correspond to a bolt (male) or other corresponding type of male threaded attachment mechanism. Some types of threaded attachment mechanisms require both the male portion and the female portion (e.g., a bolt and a nut) while other types of threaded attachment mechanisms can be used with only the male portion (e.g., a screw). “Cutting” a hole in a piece of material (e.g., a panel) can be achieved by drilling, sawing, melting with a blow torch, cutting with a laser or otherwise removing some material from the piece of material so as to create a hole. To illustrate the various embodiments the lower side of base rail unit (e.g., base rail unit 311 of FIG. 3aB) has been described as fitting on the floor of the sterilization container. However, a situation may arise where it is useful to mount the base rail unit on a wall or other surface of a sterilization container. Mounting the base rail unit on a wall or other surface of a sterilization container is considered, for the purposes herein, to be equivalent to mounting it on the floor of the sterilization container.

The phrase “at least proximate” refers to a component's (or feature's) location relative to another item. A component that is in contact with another component (or feature) is considered at least proximate. “At least proximate” can also mean that the component (or item that is at least proximate) is within a distance no greater than the largest dimension of the component itself. For example, a 1 inch long slotted hole in a universal wing adapter assembly base rail unit (e.g., FIGS. 6A-B) is at least proximate to the track rail so long as the slotted hole is within 1 inch of the track rail (since 1 inch is the largest dimension of the slotted hole, in this example). For the purposes of this disclosure, two components formed from the same piece of material are considered to be “attached”. For example, in some embodiments the universal wing adapter assembly base rail is formed from the same piece of material as the track rail by being molded or machined to shape. In such embodiments the base rail is considered to be “attached” to the track rail, even though the two components were formed from the same piece of material.

As used herein, the singular forms “a”, “an” and “the” are intended to include the plural forms as well, unless the context clearly indicates otherwise. It will be further understood that the terms “comprises,” “comprising,” “includes,” and/or “including” used in this specification, including the claims, specify the presence of stated features, integers, steps, operations, elements, and/or components, but do not preclude the presence or addition of one or more other features, integers, steps, operations, elements, components, and/or groups thereof. The terms “obtaining” or “providing”, as used herein and in the claims, means to retrieve an article or device to be assembled as part of the apparatus at issue. Further, the terms “obtaining” or “providing” may be defined to mean fabricating, or adapting another part to operate as the article or device. For example, bending up the ends of a bottom panel to form side panels can be interpreted as providing side panels attached to a bottom panel. The term “plurality”, as used herein and in the claims, means two or more of a named element. It should not, however, be interpreted to necessarily refer to every instance of the named element in the entire device. Particularly, if there is a reference to “each” element of a “plurality” of elements. There may be additional elements in the entire device that are not included in the “plurality” and are therefore, not referred to by “each.”

The corresponding structures, materials, acts, and equivalents of any means plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope or gist of the invention. The various embodiments included for discussion herein were chosen and described in order to best explain the principles of the invention and the practical application, and to enable others of ordinary skill in the art to understand the invention for various embodiments with various modifications as are suited to the particular use contemplated.

1. An apparatus for holding one or more surgical instruments securely in relation to a floor of a sterilization container, the apparatus comprising:
a base rail unit with an upper surface;
a first base rail arm configured as part of the base rail unit, the first base rail arm having at least a portion of a lower surface that fits on the floor of the sterilization container;
a track rail arranged on the base rail unit;
an adjustable bar track slidably mounted on the track rail, the adjustable bar track being configured to accept an instrument holder bar to hold the one or more surgical instruments; and
a base portion of the first base rail arm configured to accept a mechanical tightening mechanism that tightens the apparatus against the floor of the sterilization container.

2. The apparatus of claim 1, further comprising:
a second base rail arm parallel to the first base rail arm;
wherein the track rail is arranged on the upper surface of the base rail unit.

3. The apparatus of claim 1, further comprising:
a tensioner component arranged on the track rail;
wherein the tensioner component presses against the adjustable bar track.

4. The apparatus of claim 3, further comprising:
a spring within the tensioner component that causes the tensioner component to press against the adjustable bar track creating friction between the tensioner component and the adjustable bar track.

5. The apparatus of claim 2, wherein the mechanical tightening mechanism is a female threaded attachment mechanism, the apparatus further comprising:
a trough bounded by the first base rail arm and the second base rail arm, the trough having a trough width greater than a width of the female threaded attachment mechanism.

6. The apparatus of claim 5,
wherein the female threaded attachment mechanism is characterized by a distance between points parameter; and
wherein the trough width is less than the distance between points parameter of the female threaded attachment mechanism.

7. The apparatus of claim 6,
wherein the trough is a rabbet profile slot bounded by the first base rail arm and the second base rail arm; and
wherein the female threaded attachment mechanism fits into the rabbet profile slot.

8. The apparatus of claim 7, wherein the female threaded attachment mechanism is a threaded nut, the apparatus further comprising:
a male threaded attachment mechanism; and
wherein the rabbet profile slot keeps the threaded nut from turning while the male threaded attachment mechanism is being tightened or loosened.

9. The apparatus of claim 1,
wherein the apparatus has three degrees of planar positional freedom relative to the floor of the sterilization container in order to position the instrument holder bar to hold the one or more surgical instruments.

10. A method for holding one or more surgical instruments securely in relation to a floor of a sterilization container, the method comprising:
positioning a base rail unit with an upper surface on the floor of the sterilization container;
providing a first base rail arm configured as part of the base rail unit, the first base rail arm having at least a portion of a lower surface that fits on the floor of the sterilization container;
arranging a track rail on the base rail unit;
slidably mounting an adjustable bar track on the track rail, the adjustable bar track being configured to accept an instrument holder bar to hold the one or more surgical instruments; and
configuring a base portion of the first base rail arm to accept a mechanical tightening mechanism that tightens the apparatus against the floor of the sterilization container.

11. The method of claim 10, further comprising:
providing a second base rail arm parallel to the first base rail arm;
wherein the track rail is arranged on the upper surface of the base rail unit.

12. The method of claim 10, further comprising:
arranging a tensioner component on the track rail;
wherein the tensioner component presses against the adjustable bar track.

13. The method of claim 12, further comprising:
placing a spring within the tensioner component that causes the tensioner component to press against the adjustable bar track creating friction between the tensioner component and the adjustable bar track.

14. The method of claim 11, wherein the mechanical tightening mechanism is a female threaded attachment mechanism, the apparatus further comprising:
forming a trough between the first base rail arm and the second base rail arm, the trough having a trough width greater than a width of the female threaded attachment mechanism.

15. The method of claim 14,
wherein the female threaded attachment mechanism is characterized by a distance between points parameter; and
wherein the trough width is less than the distance between points parameter of the female threaded attachment mechanism.

16. The method of claim 15,
wherein the trough is a rabbet profile slot bounded by the first base rail arm and the second base rail arm; and
wherein the female threaded attachment mechanism fits into the rabbet profile slot.

17. The method of claim 16, wherein the female threaded attachment mechanism is a threaded nut, the method further comprising:
a male threaded attachment mechanism;
wherein the rabbet profile slot keeps the threaded nut from turning while the male threaded attachment mechanism is being tightened or loosened.

18. The method of claim 10,
wherein the apparatus has three degrees of planar positional freedom relative to the floor of the sterilization container in order to position the instrument holder bar to hold the one or more surgical instruments.
